(19) United States
(12) Patent Application Publication
(10) Pub. No.: US 2026/0306099 A1
(43) Pub. Date: Oct. 8, 2026

(54) SURGICAL STAPLING DEVICE WITH ARTICULATING TOOL ASSEMBLY

(71) Applicant: Covidien LP, Mansfield, MA (US)

(72) Inventor: David M. Farascioni, Ridgefield, CT (US)

(73) Assignee: Covidien LP, Mansfield, MA (US)

(21) Appl. No.: 19/478,913

(22) PCT Filed: Apr. 25, 2024
(86) PCT No.: PCT/IB2024/054046

§ 371 Date: Oct. 27, 2025

Related U.S. Application Data
(60) Provisional application No. 63/462,556, filed on Apr. 28, 2023.

(51) Int. Cl. A61B 17/072; A61B 17/00; A61B 17/29

(52) U.S. Cl. CPC A61B 17/07207; A61B 2017/00486; A61B 2017/07257; A61B 2017/07271; A61B 2017/07285; A61B 2017/2927

(57) ABSTRACT

A surgical stapling device includes an adapter assembly defining a longitudinal axis, a tool assembly that is pivotably coupled to the adapter assembly about an articulation axis that is transverse to the longitudinal axis, and a drive mechanism that includes a drive beam that is positioned distally of the articulation axis. By positioning the drive beam distally of the articulation axis, the stapling device can be fired without moving the drive beam about the articulation axis to minimize the force required to fire the stapling device.

This Application claims priority from U.S. Provisional Patent Application 63/462,556, filed 28 Apr. 2023, the entire content of which is incorporated herein by reference.

### FIELD

This disclosure is generally related to surgical stapling devices and, more particularly, to surgical stapling devices having articulating tool assemblies.

### BACKGROUND

Endoscopic stapling devices that include an elongated body and a tool assembly supported on the elongated body are well known. Typically, the tool assembly is coupled to a distal portion of the elongate body by a pivot member that defines an articulation axis that is transverse to a longitudinal axis of the elongate body to facilitate pivotable movement of the tool assembly between articulated and non-articulated positions. Such stapling devices include a drive assembly having a flexible drive beam that can bend about the articulation axis when the tool assembly is fired to eject staples from the tool assembly.

To gain better access to tissue within a body cavity during an endoscopic surgical procedure, articulation of the tool assembly over a range of ninety-degrees is desirable. However, advancing the drive assembly about the articulation axis to fire the stapling device with the tool assembly articulated to such high degrees greatly increases the firing forces required to fire the stapling device.

A continuing need exists in the art for a stapling device that can minimize the firing forces required to fire the stapling device when the tool assembly of the stapling device is in an articulated position.

### SUMMARY

This disclosure generally relates to a surgical stapling device that includes an adapter assembly defining a longitudinal axis, a tool assembly that is pivotably coupled to the adapter assembly about an articulation axis that is transverse to the longitudinal axis, and a drive mechanism that includes a drive beam that is positioned distally of the articulation axis. By positioning the drive beam distally of the articulation axis, the stapling device can be fired without moving the drive beam about the articulation axis to minimize the force required to fire the stapling device.

Aspects of the disclosure are directed to a surgical stapling device that includes an adapter assembly, a tool assembly, and a distal drive assembly. The adapter assembly has a proximal portion and a distal portion supporting a proximal drive assembly. The proximal drive assembly includes a rotatable drive member and a drive gear. The tool assembly includes a first jaw and a second jaw that are coupled to each other to facilitate movement of the tool assembly between an unclamped position and a clamped position. The tool assembly includes a proximal portion and a distal portion. The distal drive assembly includes a pivot member, a sprocket, drive plates formed of a rigid material, and a clamp member that is secured to one of the drive plates. The pivot member is secured to the proximal portion of the tool assembly. The drive plates are movable in response to rotation of the sprocket between a stacked configuration and a longitudinally aligned configuration to move the clamp member in relation to the tool assembly between retracted and advanced positions.

In aspects of the disclosure, the sprocket includes fingers and each of the drive plates defines openings that receive the fingers such that rotation of the sprocket in a first direction causes sequential advancement of the drive plates from the stacked configuration to the longitudinally aligned configuration to advance the clamp member in relation to the tool assembly.

In some aspects of the disclosure, the pivot member of the distal drive assembly supports a drive plate holder that defines a cavity the receives and supports the drive plates when the drive plates are in the stacked configuration.

In certain aspects of the disclosure, the pivot member defines a longitudinal slot that is longitudinally aligned with an inner-most one of the drive plates when the drive plates are in the stacked configuration.

In aspects of the disclosure, the pivot member is pivotably coupled in relation to the proximal drive assembly about an articulation axis that is positioned proximally of the drive plates.

In some aspects of the disclosure, the stapling device includes first and second articulation cables that extend through the adapter assembly and are coupled to the pivot member.

In certain aspects of the disclosure, the first and second articulation cables are movable to pivot the tool assembly about the articulation axis.

In aspects of the disclosure, the proximal drive assembly includes a distal housing portion that defines a circular cutout, and the pivot member supports a pivot pin that is received within the circular cutout to pivotably couple the pivot member to the proximal drive assembly.

In some aspects of the disclosure, the first and second articulation cables extend through channels in the proximal drive assembly and the pivot member, and tension in the first and second articulation cables retains the pivot pin within the circular cutout.

In certain aspects of the disclosure, the proximal drive assembly includes proximal gear that is engaged with the drive gear and the distal drive assembly includes a pivot gear that is engaged with the proximal gear such that rotation of the rotatable drive member of the proximal drive assembly causes rotation of the pivot gear of the distal drive assembly.

In aspects of the disclosure, the distal drive assembly includes a sprocket gear that is engaged with the pivot gear and the sprocket such that rotation of the rotatable drive member of the proximal drive assembly causes rotation of the sprocket of the distal drive assembly to advance the clamp member in relation to the tool assembly.

In some aspects of the disclosure, each of the drive plates includes a distally positioned rib and defines a proximally positioned opening, the ribs received within the openings of adjacent drive plates to couple the adjacent drive plates to each other as the drive plates move from the stacked configuration to the longitudinally aligned configuration.

In certain aspects of the disclosure, each of the drive plates includes an angled distal cam surface that is positioned to engage a proximal surface of an adjacent one of the drive plates to allow the drive plates to move from the longitudinally aligned position to the stacked configuration.

In aspects of the disclosure, the distal drive assembly of the stapling device includes a biasing member that is supported within the drive plate holder to urge the drive plates towards the sprocket when the drive plates are in the stacked configuration.

In some aspects of the disclosure, the stapling device includes a handle assembly, and the proximal portion of the adapter assembly is coupled to the handle assembly.

Other aspects of the disclosure are directed to a surgical stapling device that includes an adapter assembly, a tool assembly, and a distal drive assembly. The adapter assembly has a proximal portion and a distal portion. The tool assembly includes an anvil and a cartridge assembly that are coupled to each other to facilitate movement of the tool assembly between an unclamped position and a clamped position. The tool assembly includes a proximal portion and a distal portion. The distal drive assembly is secured to the proximal portion of the tool assembly and includes a clamp member, drive plates, and a pivot member pivotably coupled to the distal portion of the adapter assembly about an articulation axis. The drive plates are supported within the pivot member and are movable between a stacked configuration and a longitudinally aligned configuration to move the clamp member in relation to the tool assembly between retracted and advanced positions.

In aspects of the disclosure, the distal drive assembly includes a sprocket having fingers and each of the drive plates defines openings that receive the fingers such that rotation of the sprocket in a first direction causes sequential advancement of the drive plates from the stacked configuration to the longitudinally aligned configuration to advance the clamp member in relation to the tool assembly.

In some aspects of the disclosure, the pivot member of the distal drive assembly supports a drive plate holder that defines a cavity the receives and supports the drive plates when the drive plates are in the stacked configuration.

In certain aspects of the disclosure, a proximal drive assembly is supported on the distal portion of the adapter assembly and includes a distal housing portion that defines a circular cutout.

In aspects of the disclosure, the pivot member supports a pivot pin that is received within the circular cutout to pivotably couple the pivot member to the proximal drive assembly.

In some aspects of the disclosure, the first and second articulation cables extend through channels in the proximal drive assembly and the pivot member, and tension in the first and second articulation cables retains the pivot pin within the circular cutout.

Still other aspects of the disclosure are directed to a surgical stapling device that includes an adapter assembly, a tool assembly, and a distal drive assembly. The adapter assembly has a proximal portion and a distal portion that supports a proximal drive assembly including a rotatable drive member and a drive gear. The tool assembly includes first jaw and a second jaw that are coupled to each other to facilitate movement of the tool assembly between an unclamped position and a clamped position. The tool assembly includes a proximal portion and a distal portion. The distal drive assembly includes a pivot member, a sprocket, drive plates, and a clamp member that is secured to one of the drive plates. The pivot member is secured to the proximal portion of the tool assembly. The rigid drive plates are movable in response to rotation of the sprocket between a stacked configuration and a longitudinally aligned configuration to move the clamp member in relation to the tool assembly between retracted and advanced positions. The proximal drive assembly is supported on the distal portion of the adapter assembly and includes a distal housing portion that defines a circular cutout. The pivot member supports a pivot pin that is received within the circular cutout to pivotably couple the pivot member to the proximal drive assembly. The first and second articulation cables extend through channels in the proximal drive assembly and the pivot member such that tension in the first and second articulation cables retains the pivot pin within the circular cutout.

Other aspects, features, and advantages will be apparent from the description, the drawings, and the claims that follow.

### BRIEF DESCRIPTION OF DRAWINGS

Various aspects and features of the disclosure are described with reference to the drawings wherein like numerals designate identical or corresponding elements in each of the several views wherein:

FIG. 1 is a side perspective view of a stapling device having a tool assembly in an unclamped, non-articulated position;

FIG. 2 is an exploded view of a distal portion of the stapling device shown in FIG. 1 including the tool assembly, and a drive mechanism;

FIG. 3 is an enlarged view of the indicated area of detail shown in FIG. 2 illustrating an exploded side perspective view of a distal drive assembly of the drive mechanism and a pivot member of the stapling device shown in FIG. 1;

FIG. 3A is a perspective view from the distal end of a pivot member of the stapling device shown in FIG. 2;

FIG. 3B is a cross-sectional view taken along section line 3B-3B of FIG. 3A;

FIG. 4 is an enlarged view of the indicated area of detail shown in FIG. 1;

FIG. 5 is an enlarged view of the indicated area of detail shown in FIG. 4;

FIG. 6 is a side perspective view of the distal portion of the adapter assembly of the stapling device shown in FIG. 1 with an outer tube of the adapter assembly removed;

FIG. 7 is a cross-sectional view taken along section line 7-7 of FIG. 6;

FIG. 8 is a side perspective view of a drive sprocket and I-beam assembly of the distal drive assembly of the drive mechanism of the stapling device shown in FIG. 1 with the drive sprocket separated from the I-beam assembly;

FIG. 9 is a side perspective view of the I-beam assembly of the distal drive assembly of the drive mechanism shown in FIG. 8;

FIG. 10 is a side perspective view of a drive plate of the I-beam assembly shown in FIG. 9;

FIG. 11 is a cross-sectional view taken along section line 11-11 of FIG. 13;

FIG. 12 is an exploded side perspective view of a distal portion of the adapter assembly including a proximal drive assembly of the drive mechanism of the stapling device shown in FIG. 1;

FIG. 13 is a cross-sectional view taken along section line 13-13 of FIG. 5;

FIG. 14 is a side perspective view of a distal portion of the stapling device shown in FIG. 1 with the tool assembly in an articulated, unclamped position;

FIG. 15 is a cross-sectional view taken along section line 15-15 of FIG. 14;

FIG. 16 is a plan view of a proximal portion of the tool assembly and the drive mechanism of the stapling device shown in FIG. 1 with the tool assembly in a clamped and articulated position;

FIG. 17 is a cross-sectional view taken along section line 17-17 of FIG. 15;

FIG. 18 is a plan view of the proximal portion of the tool assembly and the drive mechanism shown in FIG. 16 with the tool assembly in the clamped and articulated position as the I-beam assembly is advanced through the tool assembly during firing of the stapling device;

FIG. 19 is an enlarged view of the indicated area of detail shown in FIG. 18;

FIG. 20 is a cross-sectional view taken through the tool assembly of the stapling device shown in FIG. 1 with the tool assembly in an articulated position and the I-beam assembly of the distal drive assembly of the drive mechanism in an advanced position after the stapling device is fired;

FIG. 21 is a cross-sectional view taken along section line 21-21 of FIG. 20;

FIG. 22 is an enlarged view of the indicated area of detail shown in FIG. 21;

FIG. 23 is a cross-sectional view taken through the proximal portion of the tool assembly and the distal drive assembly of the drive mechanism with the I-beam assembly of the distal drive assembly of the drive mechanism in an advanced position after the stapling device is fired;

FIG. 24 is a cross-sectional view taken through the tool assembly of the stapling device shown in FIG. 1 with the tool assembly in an articulated position as the I-beam assembly of the distal drive assembly of the drive mechanism is moved from the advanced position towards the retracted position after the stapling device is fired;

FIG. 25 is a cross-sectional view taken through the proximal portion of the tool assembly and the distal drive assembly of the drive mechanism of the stapling device shown in FIG. 1 as the I-beam assembly of the distal drive assembly of the drive mechanism is moved from the advanced position towards the retracted position after the stapling device is fired; and

FIG. 26 is a cross-sectional view taken through the proximal portion of the tool assembly and the distal drive assembly of the drive mechanism of the stapling device shown in FIG. 1 as the I-beam assembly of the distal drive assembly of the drive mechanism is moved from the position shown in FIG. 25 towards the retracted position.

### DETAILED DESCRIPTION

The disclosed surgical stapling device will now be described in detail with reference to the drawings in which like reference numerals designate identical or corresponding elements in each of the several views. However, it is to be understood that aspects of the disclosure included herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device for its intended purpose, while the term “distal” is used generally to refer to that portion of the device that is farther from the clinician during use of the device for its intended purpose. In addition, the term “clinician” is used generally to refer to medical personnel including doctors, nurses, and support personnel, and directional terms such as front, rear, upper, lower, top, bottom, and similar terms are used to assist in understanding the description and are not intended to limit the disclosure.

This disclosure is directed to a surgical stapling device that includes an adapter assembly defining a longitudinal axis, a tool assembly that is pivotably coupled to the adapter assembly about an articulation axis that is transverse to the longitudinal axis, and a drive mechanism that includes a drive beam that is positioned distally of the articulation axis. By positioning the drive beam distally of the articulation axis, the stapling device can be fired without moving the drive beam about the articulation axis to minimize the force required to fire the stapling device.

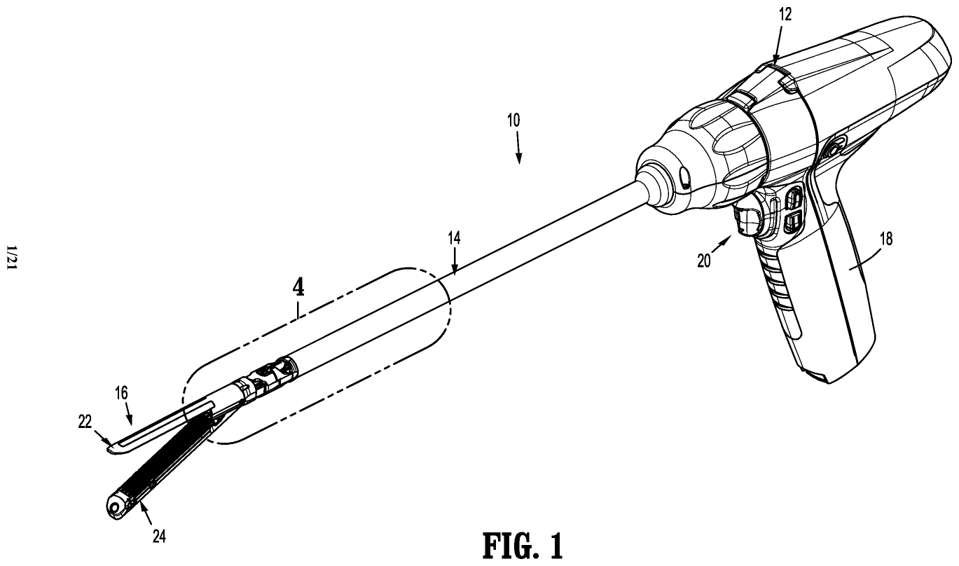

FIG. 1 illustrates a surgical stapling device shown generally as stapling device 10 that includes a handle assembly 12, an elongated body or adapter assembly 14, and a tool assembly 16. In aspects of the disclosure, the handle assembly 12 is powered and includes a stationary handgrip 18 and an actuation button or buttons 20. The actuation buttons 20 are operable to actuate various functions of the tool assembly 16 via the adapter assembly 14 including approximation, stapling, cutting of tissue, and articulation of the tool assembly 16. Although not shown, the handle assembly 16 can support batteries (not shown) that provide power to the handle assembly 12 to actuate the stapling device 10.

The tool assembly 16 is supported on a distal portion of the adapter assembly 14 as described in further detail below and includes a first jaw, e.g., an anvil 22 and a second jaw, e.g., a cartridge assembly 24. Although the stapling device 10 is illustrated as an electrically powered hand held stapling device, it is envisioned that the advantages of this disclosure are suitable for use with manually powered surgical stapling devices. In addition, it is contemplated, and within the scope of the disclosure for the stapling device to be configured for use with various electromechanical and/or electrosurgical instruments and systems. For example, the stapling device may be utilized in robotic surgical systems, such as the robotic surgical system shown and described in U.S. Pat. No. 8,828,023, the entire content of which is incorporated herein by reference.

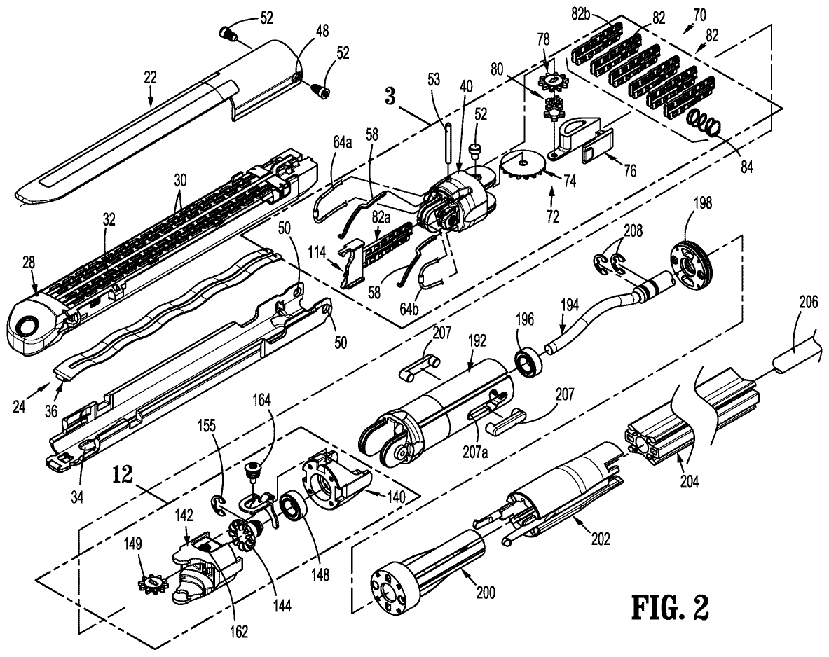

FIG. 2 illustrates the cartridge assembly 24 which includes a channel member 26 and a staple cartridge 28 that is supported within the channel member 26. The staple cartridge 28 defines staple receiving pockets 30 (FIG. 2) and a knife slot 32. The staple receiving pockets 30 are aligned in rows on opposite sides of the knife slot 32 and receive staples (not shown). The channel member 26 also defines a knife slot 34 that is longitudinally aligned with the knife slot 32 in the staple cartridge 28. In aspects of the disclosure, the cartridge assembly 24 includes a biasing member 36 that is supported on a bottom wall of the channel member 26 and supports the staple cartridge 28. The biasing member 36 is compressible or deformable to facilitate vertical movement of the staple cartridge 28 within channel member 26 to accommodate tissue of varying thicknesses. The biasing member 36 can be in the form of a wave spring or a compliant pad, although the use of other compressible or deformable materials is envisioned.

The tool assembly 16 is fixedly secured to a pivot member 40 that is positioned proximally of the tool assembly 16. The pivot member 40 includes a body 42 that defines a longitudinal slot 44 and threaded bores 46. The longitudinal slot 44 extends through the body 42 of the pivot member 40 and the threaded bores 46 are positioned on opposite sides of the longitudinal slot 44. In aspects of the disclosure, the proximal portion of the anvil 22 defines openings 48 and the proximal portion of the channel member 26 of the cartridge assembly 22 defines openings 50. The openings 48 and 50 receive screws 52 that fixedly secure the anvil 22 to the pivot member 40 and pivotably secure the channel member 26 to the pivot member 40 to facilitate movement of the channel member 26 in relation to the anvil 22 between an unclamped position and a clamped position.

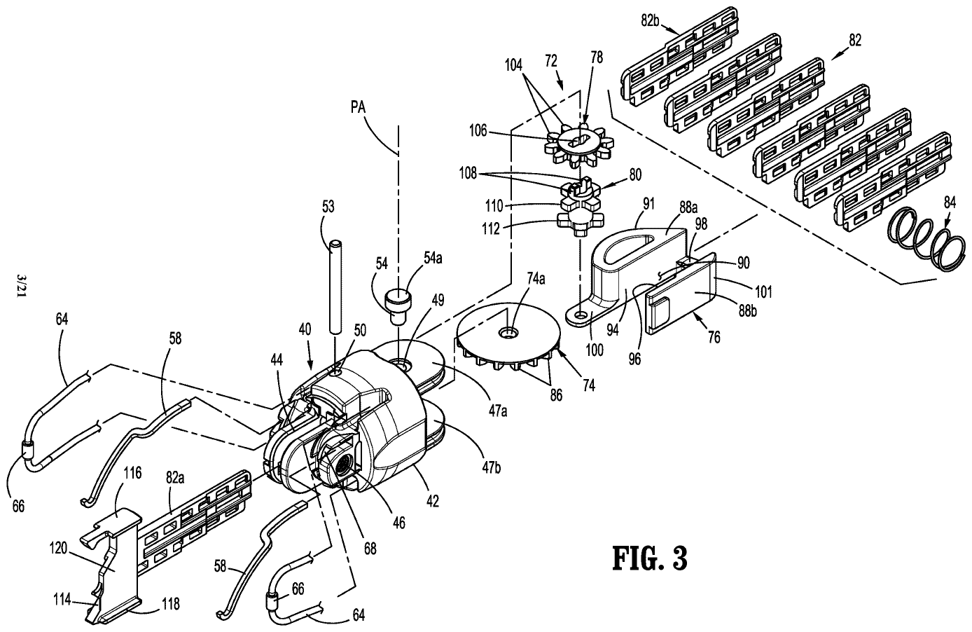

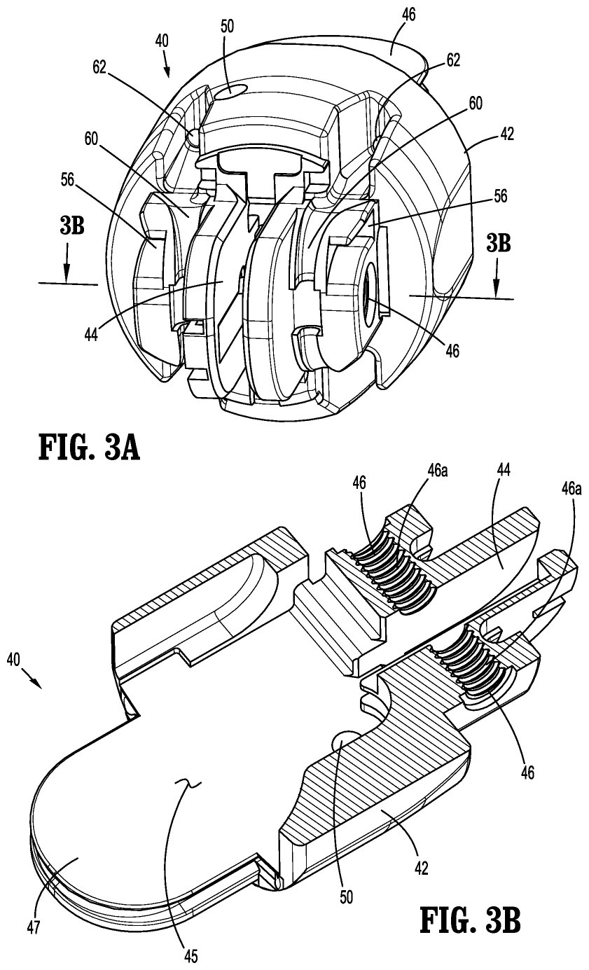

FIGS. 3-3B illustrate the pivot member 40 which is secured to the proximal portion of the tool assembly 16 (FIG. 1) and includes the body 42. The body 42 defines an inner cavity 45 that communicates with the longitudinal slot 44 and includes proximally extending hinges including an upper hinge 47a and a lower hinge 47b as viewed in FIG. 3. The body 42 of the pivot member 40 also defines a bore 50 that extends through the body 42 and the inner cavity 45 (FIG. 3B) in a direction that is transverse to an axis defined by the threaded bores 46. The bore 42 receives an elongated pin 53. The upper hinge 47a defines a bore 49 that receives a pivot pin 54 that includes a head portion 54a that has a diameter larger than a body of the pivot pin 54. The body 42 of the pivot member 40 has a distal portion that defines spaced recesses 56 that are positioned on opposite sides of the longitudinal slot 44 adjacent the threaded bores 46. Each of the spaced recesses 56 receive a proximal portion of a respective biasing member 58 to secure the biasing member 58 to the pivot member 40. In aspects of the disclosure, the biasing members 58 are formed of a resilient material, e.g., spring steel, and include a distal portion that is engaged with the channel member 26 of the cartridge assembly 24 to urge the cartridge assembly towards the unclamped position.

The body 42 of the pivot member 40 also defines curved tracks 60 that are positioned on opposite sides of the longitudinal slot 44. The curved tracks 60 are aligned with guide bores 62 (FIG. 3A) that extend through the proximal portion of the body 42 and receive articulation cables 64a, 64b that are positioned on opposite sides of the longitudinal slot 44. The articulation cables 64a, 64b extend about the respective curved tracks 60, through the upper and lower guide bores 62, and proximally through the adapter assembly 14 to the handle assembly 12 (FIG. 1). The articulation cables 64a, 64b are movable to facilitate articulation of the pivot member 40 and tool assembly 16 in relation to the adapter assembly 14 as described in further detail below. In aspects of the disclosure, the articulation cables 64a, 64b support ferrules 66 (FIG. 3) that are received within slots 68 (FIG. 3) formed along the curved tracks 60 to secure the articulation cables 64 to the pivot member 40.

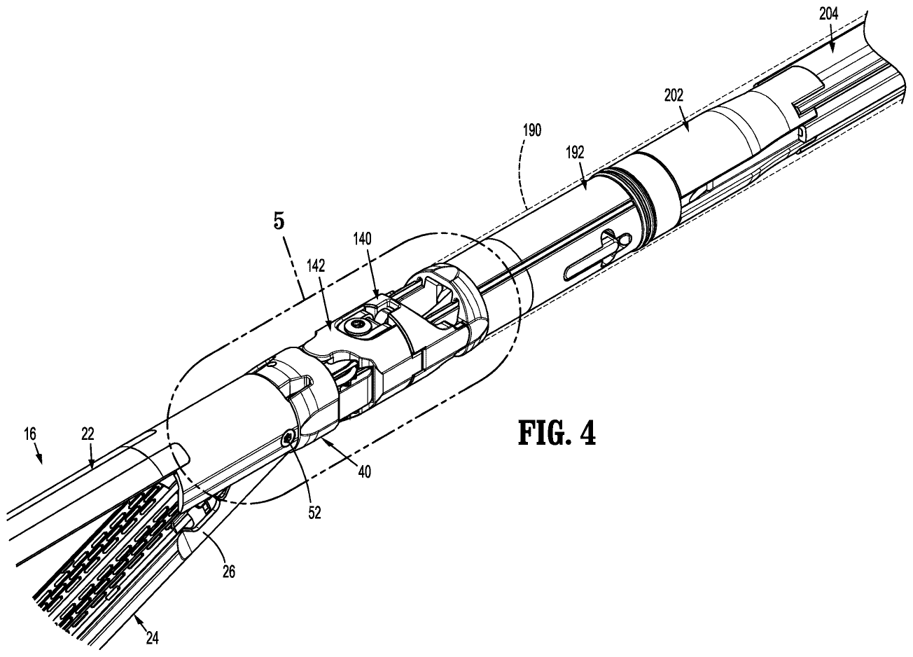

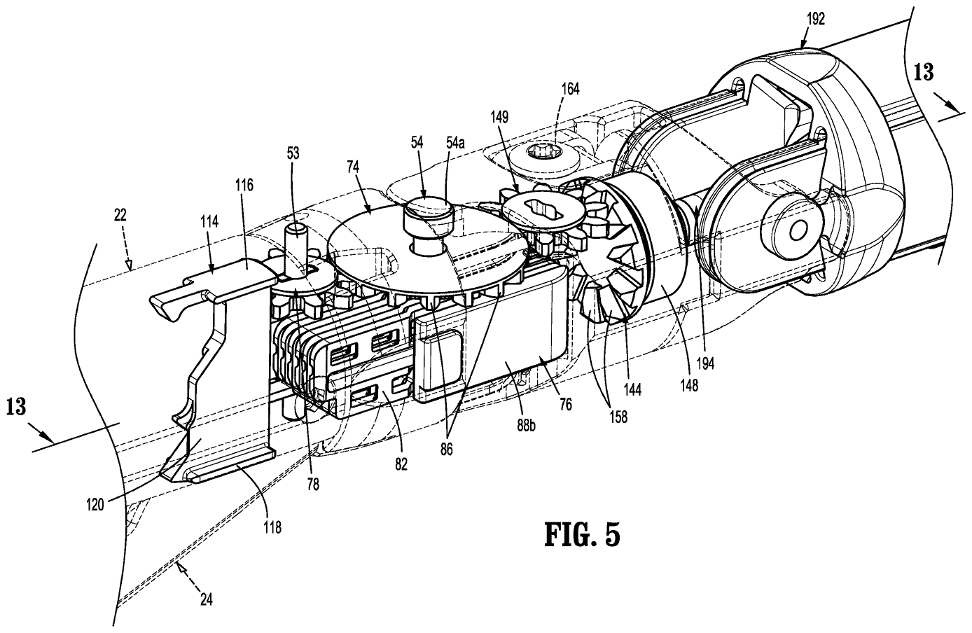

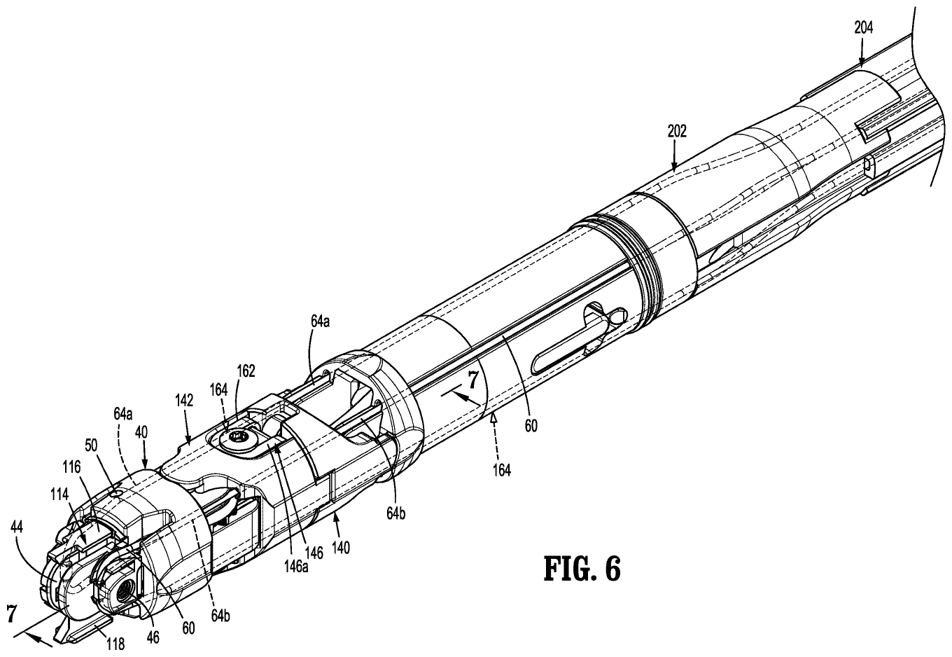

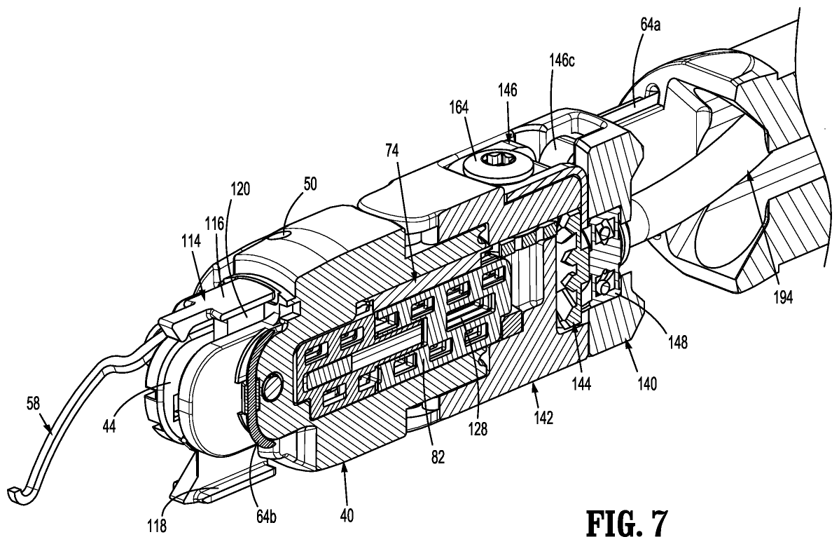

FIGS. 3-7 illustrate a distal drive assembly 72 of a drive mechanism 70 (FIG. 2) of the stapling device 10 (FIG. 1). The distal drive assembly 72 is supported on the pivot member 40 and includes a clamp member 114, a pivot gear 74, a drive plate holder 76, a sprocket gear 78, a sprocket 80, drive plates 82, and a biasing member 84. The pivot gear 74 has a circular configuration with a central bore 74a and includes a plurality of gear teeth 86 positioned about the circumference of the pivot gear 74. The central bore 74a of the pivot gear 74 receives the pivot pin 54 to rotatably couple the pivot gear 74 to the upper hinge 47a of the pivot member 40 at a proximal end of the pivot member 40 between the upper and lower hinges 47a, 47b.

The drive plate holder 76 includes a body 88 defining a cavity 90 for receiving the drive plates 82. The body 88 has a first portion 88a having a curved outer surface 91 and a linear or flat inner surface 94, and a second portion 88b having a linear or flat inner surface 96 that is spaced from the inner surface 94 of the first portion 88a to define the cavity 90. The first portion 88a of the drive plate holder 76 is connected to the second portion 88b by a spacer 98 that is positioned at the proximal portion of the drive plate holder 76. The first portion 88a of the drive plate holder 76 includes a distally extending flange 100 that defines a bore 102 (FIG. 3) that receives the elongated pin 53 to secure the drive plate holder 76 to the pivot member 40 such that the drive plate holder 76 extends proximally from the cavity 45 of the pivot member 40. The proximal end of the second portion 88b of the drive plate holder 76 also has a curved surface 101.

The sprocket gear 78 and the sprocket 80 are rotatably supported within the cavity 45 of the pivot member 40 about the elongated pin 53. The sprocket gear 78 includes gear teeth 104 that are engaged with the gear teeth 86 of the pivot gear 74 such that rotation of the pivot gear 74 causes rotation of the sprocket gear 78. The sprocket gear 78 is rotatably fixed to the sprocket 80 to translate rotation of the sprocket gear 78 into rotation of the sprocket 80. In aspects of the disclosure, the sprocket gear 78 defines a rectangular slot 106 (FIG. 3) and the sprocket 80 includes a pair of fingers 108 that are received within the rectangular slot 106 to secure the sprocket gear 78 to the sprocket 80. The sprocket 80 includes an annular array of upper and lower fingers 110, 112 that project outwardly from the sprocket 80 to a position slightly distal of the cavity 90 defined by the drive plate holder 76. The fingers 110, 112 engage the inner most drive plate 82a of a stack of drive plates 82 supported within the cavity 90 of the drive plate holder 76 as described in further detail below. The drive plates 82 are rigid and releasably connectable to adjacent drive plates 82 to form a drive beam as described below. The inner-most drive plate 82a is aligned with the longitudinal slot 44 of the pivot member 40.

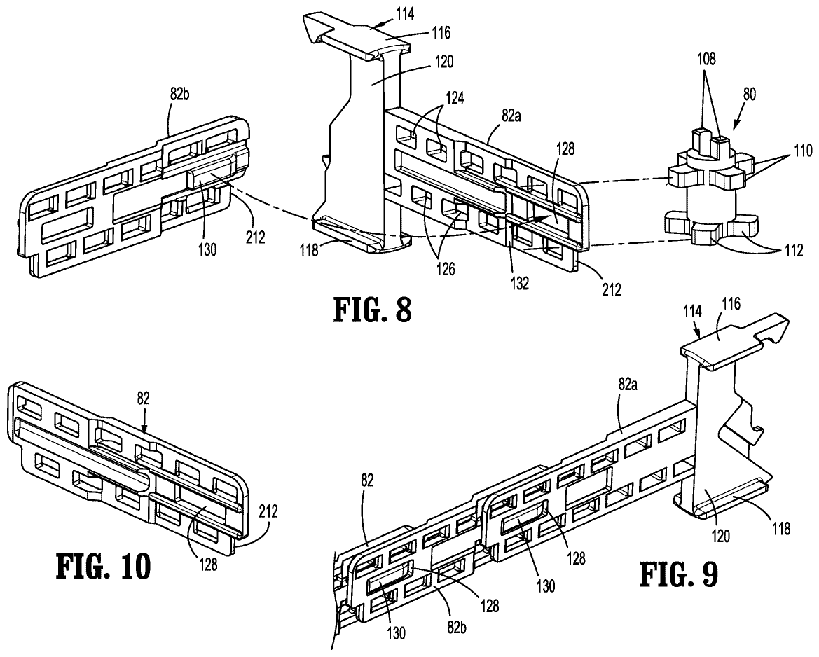

FIGS. 8-11 illustrate the clamp member 114 and drive plates 82 of the distal drive assembly 72 of the drive mechanism 70. The clamp member 114 is fixedly secured to the inner-most drive plate 82a and includes a body that has an I-shaped configuration with a first beam 116, a second beam 118, and a vertical strut 120 that fixedly connects the first beam 116 to the second beam 118. The first beam 116 is positioned to engage the anvil 22 and the second beam 118 is positioned to engage the channel member 26 of the cartridge assembly 24 as the drive mechanism 70 is moved from a drive retracted position towards a drive advanced position to move the tool assembly 16 (FIG. 1) from the unclamped position to the clamped position.

The clamp member 114 and the drive plates 82 of the distal drive assembly 72 of the drive mechanism 70 are supported on their end walls within the cavity 90 of the drive plate holder 76 between the inner surfaces 94, 96 of the drive plate holder 76 in stacked fashion. In the stacked position, the drive plates 82 extend through a plane that is substantially the longitudinal axis defined by the longitudinal slot 44 of the pivot member 40. In aspects of the disclosure, each of the drive plates 82 has a rectangular configuration and defines an upper row of openings 124, a lower row of openings 126, a recess or central opening 128 that is positioned between the upper and lower openings 124, 126 on a proximal portion of the drive plate 82, and a rib 130 that is positioned on the distal portion of the drive plate 82. The upper and lower openings 124, 126 receive the upper and lower fingers 110, 112 of the sprocket 80 such that the drive plates 82 can be sequentially advanced through the longitudinal slot 44 of the pivot body 40 to advance the clamp member 114 through the tool assembly 16 (FIG. 1). The ribs 130 on the distal portions of the drive plates 82 are configured to be received within the openings 128 in the proximal portions of adjacent drive plates 82 to couple the adjacent drive plates 82 to each other when the drive mechanism 70 is moved from the drive retracted position towards the drive advanced position as described in further detail below. In aspects of the disclosure, each of the drive plates 82 has a side wall with a stepped configuration such that the distal portion of a proximal drive plate 82 is received within a proximal portion of an adjacent distally positioned drive plate 82.

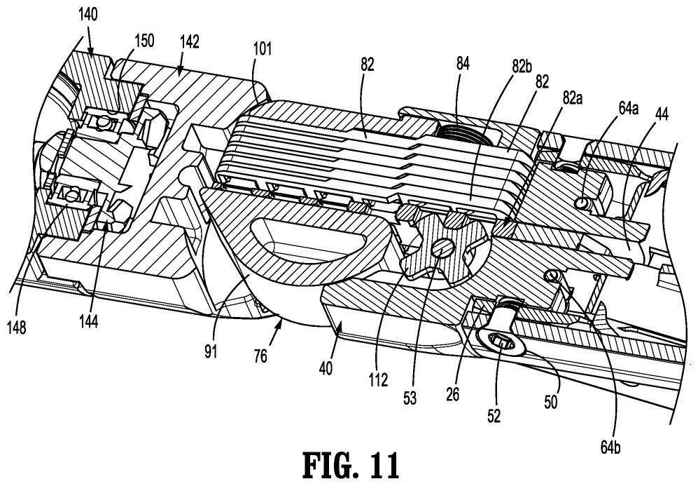

When the drive mechanism 70 is in a retracted position (FIG. 11), the vertical strut 120 of the clamp member 114 is received within the longitudinal slot 44 of the pivot member 40 and the inner-most drive plate 82a which is secured to the clamp member 114 extends proximally from the clamp member 114 into the cavity 45 of the pivot member 40. In this position, the upper and lower fingers 110, 112 of the sprocket 80 are received within the upper and lower openings 124, 126 of the drive plate 82a. When the sprocket 80 is rotated as described in further detail below, rotation of sprocket 80 rotates the fingers 110, 112 sequentially into the openings 124, 126 of the drive plate 82a to advance the drive plate 82a and the clamp member 114 from the retracted position towards the advanced position. As the inner-most drive plate 82a is advanced from within the cavity 90 of the drive plate holder 76, the biasing member 84 (FIG. 3) urges the stack of drive plates 82 inwardly within the cavity 90 of the drive plate holder 76 to position distal portion of the second drive plate 82b (FIG. 9) of the stack of drive plates 82 into engagement with the proximal portion of the drive plate 82a. When the drive plate 82b engages the drive plate 82a, the rib 130 of the drive plate 82b is received in the opening 128 of the drive plate 82a to couple the drive plates 82a and 82b together. This process continues as the drive mechanism 70 moves from the drive retracted position to the drive advanced position until each of the drive plates 82 is removed and advanced from the stack of drive plates 82.

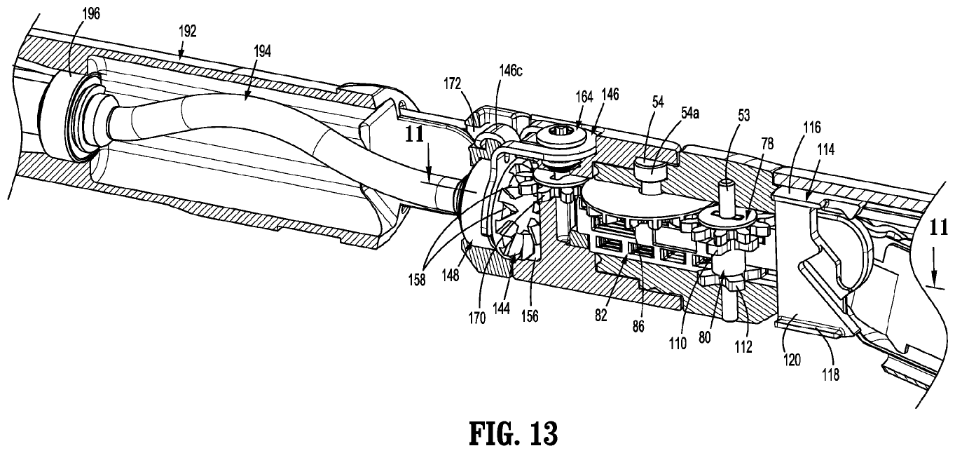

FIGS. 11-13 illustrate a proximal drive assembly 134 of the drive mechanism 70. The proximal drive assembly 134 forms the distal portion of the adapter assembly 14 (FIG. 1) and includes a proximal housing portion 140, a distal housing portion 142, a drive gear 144, a bracket 146, and a proximal gear 149. The proximal housing portion 140 defines a stepped through bore 150 that receives the bearing 148. The drive gear 144 includes a gear plate 152 and a shaft portion 154 that extends proximally from the gear plate 152. The shaft portion 154 of the drive gear 144 is rotatably supported within a bore 148a defined by the bearing 148 and extends proximally from the stepped through bore 150 of the proximal housing portion 140 of the proximal drive assembly 134. The shaft portion 154 defines an annular slot 154a that receives a clip 155 to secure the shaft 154 to a proximal side of the bearing 148. The gear plate 152 of the drive gear 144 is received within a cavity 156 (FIG. 13) defined by the distal housing portion 142 of the proximal drive assembly 134 of the drive mechanism 70 and includes distally facing teeth 158. The distal housing portion 142 of the proximal drive assembly 134 also defines an outer surface defining a recess 160 and a threaded bore 162 that is positioned within the recess and communicates with the cavity 156 (FIG. 13).

The bracket 146 includes a U-shaped portion 146a that is secured within the recess 160 of the distal housing portion 142 of the proximal drive assembly 134 of the drive mechanism 70 by a screw 164 that is received within the threaded bore 162 of the distal housing portion 142. The screw 164 includes a head portion 166 and a pivot pin 168 that extends downwardly from the head portion 166 into the cavity 156. The proximal gear 149 is rotatably supported on the pivot pin 168 of the screw 164 and includes teeth 170 that are engaged with the distally facing teeth 158 of the drive gear 144 of the distal drive assembly 72 of the drive mechanism 70 such that rotation of the drive gear 144 causes rotation of the proximal gear 149.

The bracket 146 includes a C-shaped member 146b that extends downwardly from the U-shaped portion 146a and is received about the shaft portion 154 of the drive gear 144 to retain the drive gear 144 at a fixed position within the cavity 156 of the distal housing portion 142 of the proximal drive assembly 134. The bracket 146 also includes a hook portion 146c that is received within a cutout 172 of the proximal housing portion 140 of the proximal drive assembly 134 to secure the proximal housing portion 140 to the distal housing portion 142.

The distal housing portion 142 of the proximal drive assembly 134 includes upper and lower flanges 174a, 174b that are spaced from each other and are positioned on outer surfaces of the upper and lower flanges 47a, 47b when the proximal drive assembly 134 is coupled to the pivot member 40. The distal housing portion 142 includes a circular distal surface 178 that is positioned adjacent the curved outer surfaces of the 91, 101 of the drive plate holder 76 to facilitate rotatable movement of the pivot member 40 in relation to the distal housing portion 142 of the proximal drive assembly 134. The lower flange 174a of the distal housing portion 142 includes an inner surface that defines a circular cutout 180 that receives the head portion 54a of the pivot pin 54 which is secured to the upper hinge 47a of the pivot member 40. Receipt of the head portion 54a of the pivot pin 54 within the circular cutout 180 of the lower flange 174a of the distal housing portion 142 of the proximal drive assembly 134 pivotably couples the pivot member 40 to the proximal drive assembly 134 of the drive mechanism 70 with the pivot pin 54 defining the articulation axis “PA” (FIG. 3).

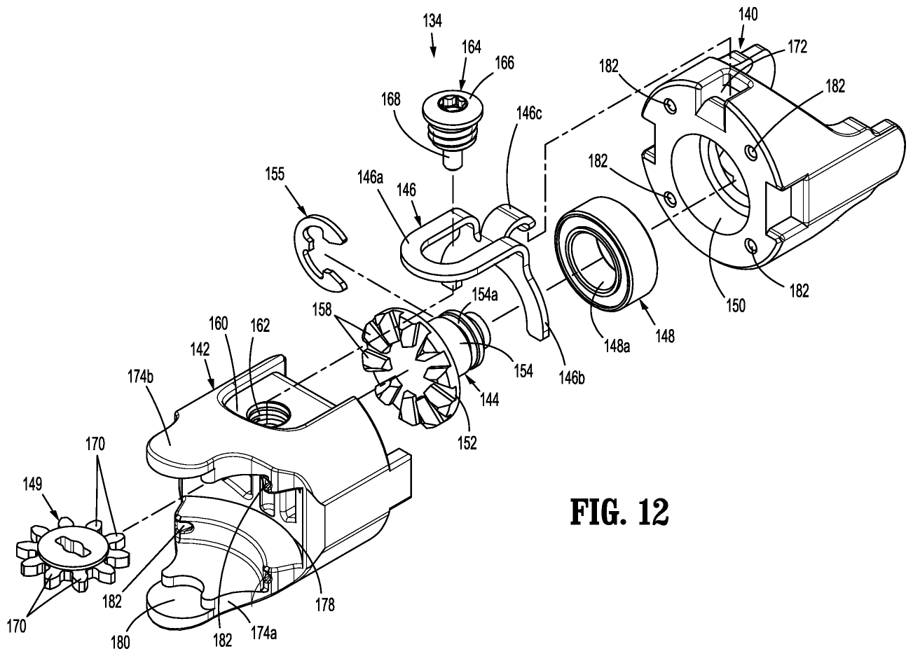

The distal housing portion 142 and the proximal housing portion 140 define upper and lower guide bores 182 (FIG. 12) that facilitate passage of the articulation cables 64 (FIG. 3) through the proximal drive assembly 134 into the adapter assembly 14 (FIG. 1). Tension in the articulation cables 64a, 64b retains the head portion 54a of the pivot pin 54 within the circular cutout 180 of the lower flange 174a of the distal housing portion 142 to pivotably couple the proximal drive assembly 134 to the pivot member 40. As described above, the tool assembly 16 (FIG. 1) is secured to the pivot member 40 such that the tool assembly 16 is pivotably supported on the distal portion of the adapter assembly 14 (FIG. 1) about the articulation axis “PA” defined by the pivot pin 54. When the pivot member 40 is coupled to the proximal drive assembly 134, the teeth 170 of the proximal gear 149 engage the teeth 74 of the pivot gear 74 of the distal drive assembly 72 such that rotation of the proximal gear 149 cause rotation of the pivot gear 74.

FIGS. 1 and 2 illustrate components of the adapter assembly 14 (FIG. 1) which include an outer tube 190 (FIG. 1), a coupling member 192, a distal flexible drive member 194, bearings 196 and 198, an inner cable guide seal 200, an outer cable guide seal 202, a cable guide member 204, and a proximal flexible proximal drive member 206. The distal flexible drive member 194 is rotatably supported between the bearings 196 and 198 within the coupling member 192 and includes a proximal portion that is coupled to a distal portion of the proximal flexible drive member 206. The bearing 198 is secured to the proximal portion of the distal flexible drive member 194 by spaced C-clips 208. The bearing 198 is also retained in a fixed position within the coupling member 192 by radial bearing locks 207 that extend through slots 192a in the coupling member 192. The inner cable guide seal 200, outer cable drive seal 202, and the cable guide member 204 are supported within the distal portion of the outer tube 190 of the adapter assembly 14 (FIG. 1) and function to align the articulation cables 64a, 64b within the adapter assembly 14 and seal the distal portion of the adapter assembly 14. The flexible drive member 194 is secured to the shaft portion 154 of the drive gear 144 to cause selective rotation of the drive gear 144. When the drive gear 144 is rotated by the distal flexible drive member 194, the drive gear 144 rotates the proximal gear 149. As described above, the proximal gear 149 is engaged with the pivot gear 74 which is engaged with the sprocket gear 78. The sprocket gear 78 drives the sprocket 80 and the sprocket 80 advances and retracts the drive plates 82.

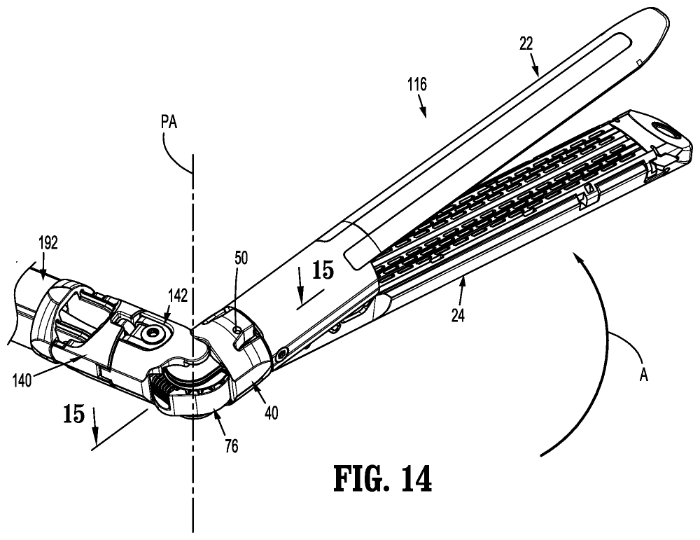

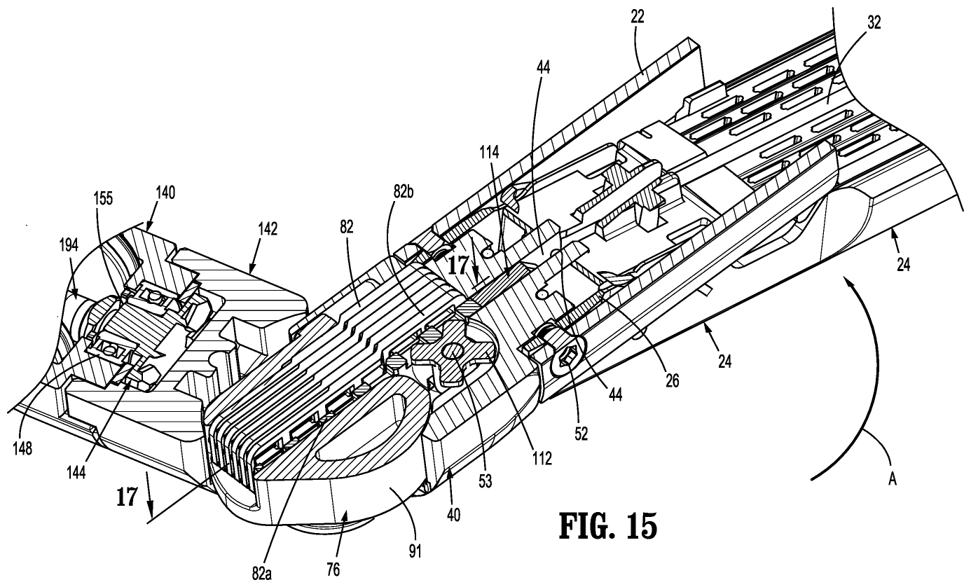

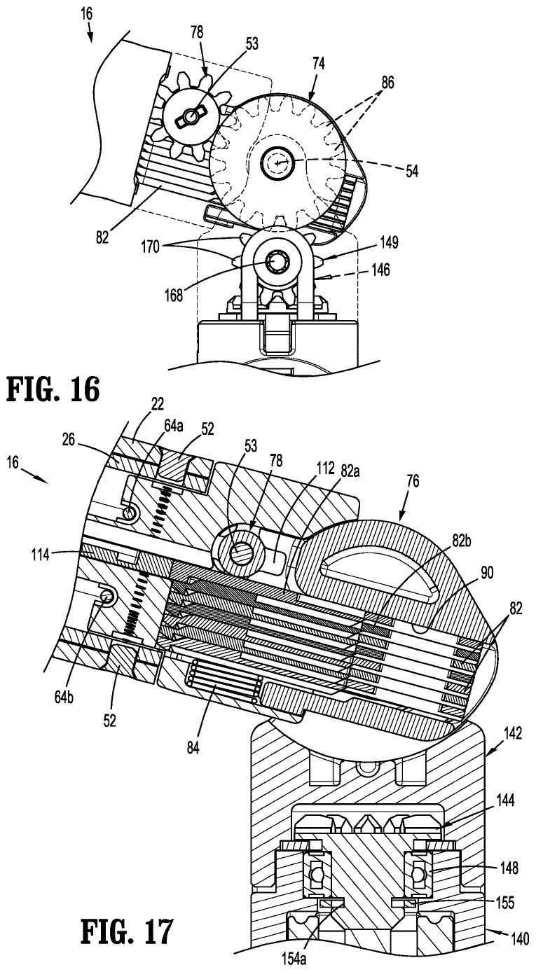

FIGS. 14-17 illustrate the stapling device 10 (FIG. 1) as the tool assembly 16 is moved to the articulated position. When the articulation cables 64a and 64b are retracted and advanced, respectively, the pivot member 40 which supports the tool assembly 16 pivots in the direction indicated by arrow “A” in FIG. 14 about the articulation axis “PA” from the non-articulated position to an articulated position. When the tool assembly 16 is in an articulated position, the distal drive assembly 72 of the drive mechanism 70 is positioned distally of the articulation axis “PA”. As illustrated, when the tool assembly 16 is in an articulated position, the proximal gear 149 of the proximal drive assembly 134 is engaged with the pivot gear 74 of the distal drive assembly 72.

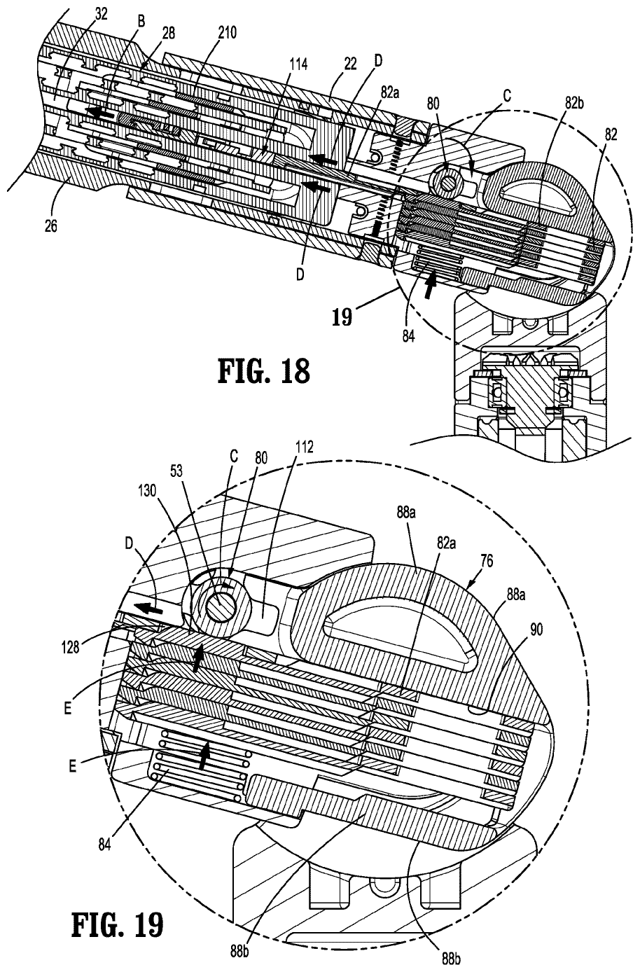

FIGS. 18 and 19 illustrate the tool assembly 16 of the stapling device 10 (FIG. 1) as the stapling device 10 is fired and the drive plates 82 are advanced to advance the clamp member 114 through the tool assembly 16 in the direction of arrows “B”. The staple cartridge 28 includes an actuation sled 210 that is driven by the clamp member 114 to eject staples (not shown) from the staple cartridge 28 as the clamp member 114 is driven through the tool assembly 16. As described above, the fingers 110, 112 of the sprocket 80 are received within the upper openings 124 and lower openings 126 and engage the drive plates 82 as the sprocket 80 is rotated in the direction indicated by arrow “C” in FIG. 19 to advance the drive plates 82 in the direction indicated by arrow “D” distally within the knife slot 32 of the staple cartridge 28. When the drive plates 82a, 82 are advanced in the direction of arrow “D”, the biasing member 84 urges the next inner-most drive plate 82 in the direction of arrow “E” into engagement with the fingers 110, 112 of the sprocket 80 to sequentially advance the drive plates 82a, 82b and 82 into the tool assembly 16 and move the clamp member 114 to the advanced position. As the drive plates 82a, 82b, and 82 are advanced, the 130 are received within the opening 128 of a proximally positioned drive plate 82 to couple the drive plates 82 together to form a substantially rigid drive beam.

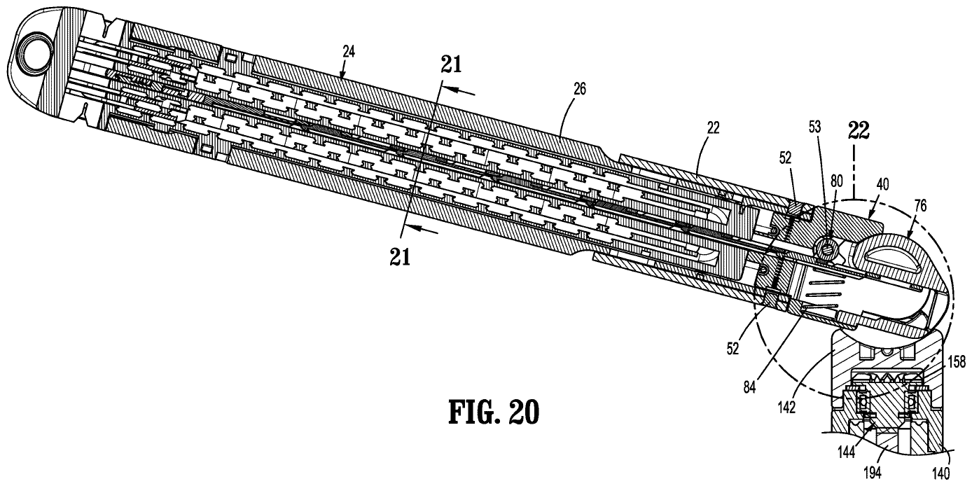

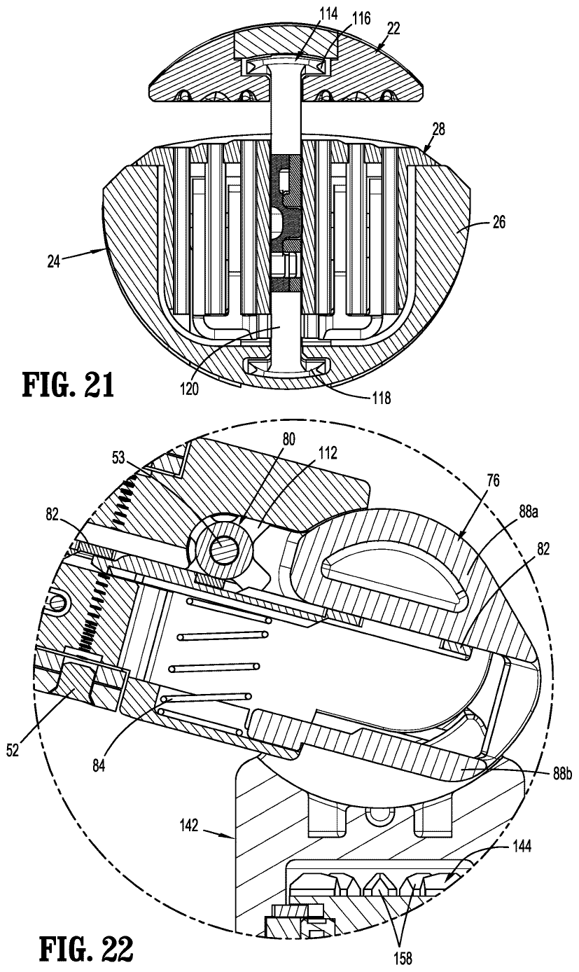

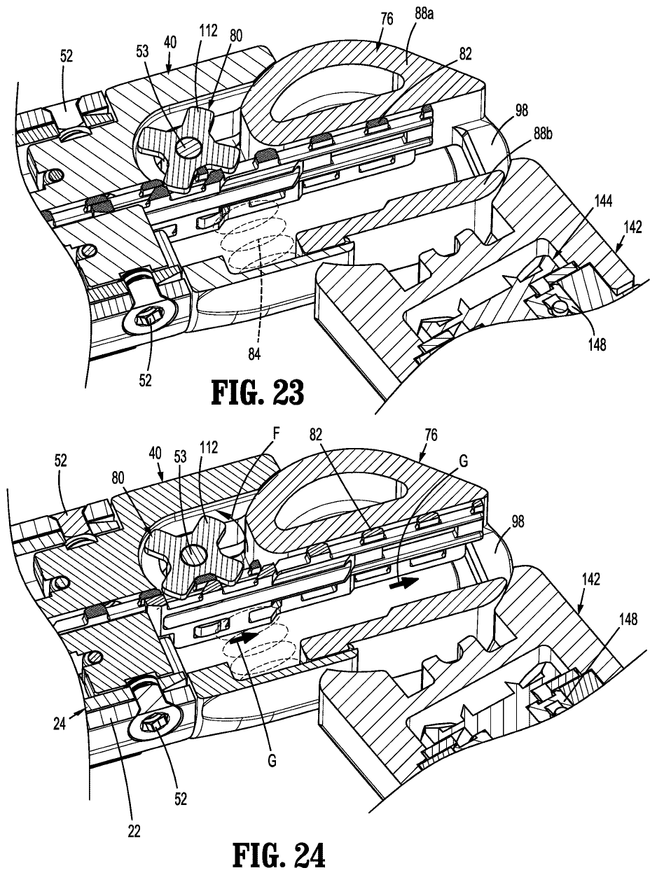

FIGS. 20-23 illustrate the tool assembly 16 with the drive mechanism 70 in the drive advanced position. When the drive mechanism 70 is moved to the drive advanced position, the proximal-most drive plate 82 is urged by the biasing member 84 into engagement with the sprocket 80 of the distal drive assembly 72 of the drive mechanism 70 and the clamp member 114 is positioned in the distal portion of the tool assembly 114.

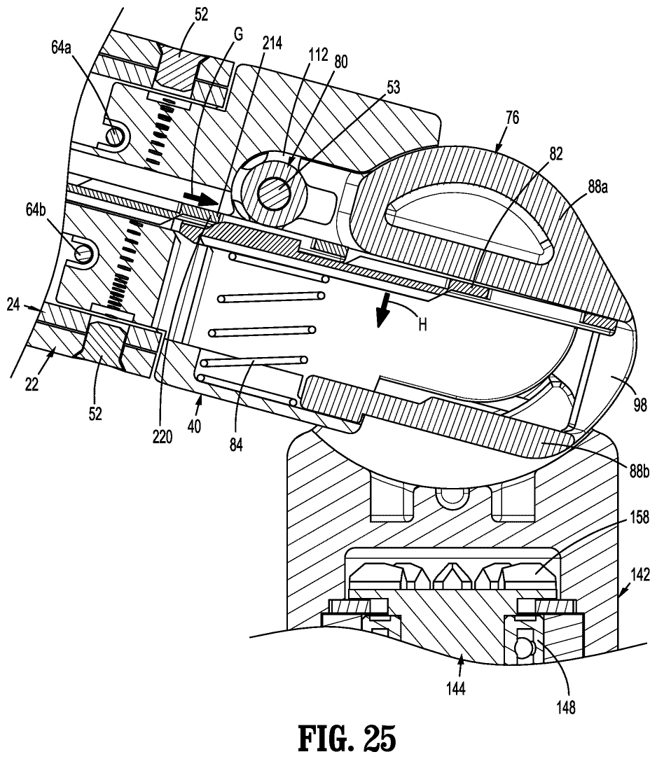

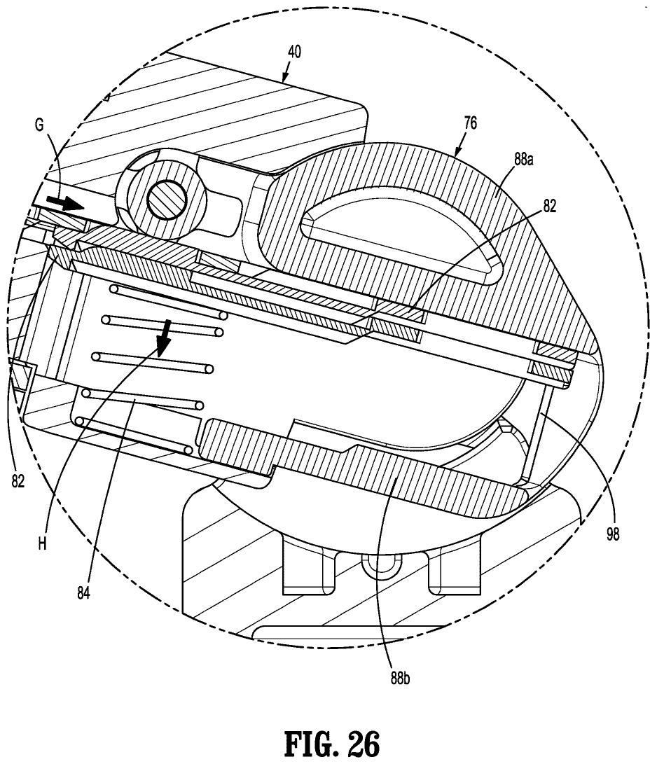

FIGS. 24-26 illustrate the distal drive assembly 72 as the drive mechanism 70 moves from the drive advanced position towards the drive retracted position. When the sprocket 80 is rotated in the direction indicated by arrow “F” in FIG. 24, the upper and lower fingers 110, 112 of the sprocket 80 engage the proximal-most drive plate 82 and pull the drive plates 82 proximally in the direction indicated by arrow “G” in FIG. 25 to move the clamp member 114 towards the retracted position. As each drive plate 82 is returned to the cavity 90 defined by the drive plate holder 76, the proximal portion of each of the drive plates 82 engages the spacer 98 of the drive plate holder 76 to prevent further proximal movement of the drive plates 82. In some aspects of the disclosure, the proximal portion of each of the drive plates 82a, 82b, and 82 defines a notch 212 (FIG. 8) that receives the spacer 98 of the drive plate holder 76 to define the fully retracted position of the drive plates 82a, 82b, and 82 within the cavity 90 of the drive plate holder 76. In aspects of the disclosure, each of the drive plates 82 includes an angled distal cam surface 214 (FIG. 25) that allow the drive plates 82a, 82b, and 82 to return to a stacked configuration within the cavity 90 of the drive plate holder 76. When the drive plates 82a, 82b, and 82 are received within the drive plate holder 76, the angled distal cam surfaces 214 of the drive plates 82a, 82b, and 82 engage the proximal portion 220 of a distally positioned drive plate 82 to allow the distally positioned drive plates 82 to move inwardly of the inner-most drive plate 82 into the stacked configuration in the direction indicated by arrow “H” in FIG. 25 against the urging of the biasing member 84.

The drive mechanism 70 described herein positions the drive beam distally of the articulation axis “PA” defined between the tool assembly 16 and the adapter assembly 14. Thus, the drive beam formed by the drive plates 82 does not have to bend about the articulation axis “PA” during firing to minimize the force required to fire the stapling device 10 (FIG. 1).

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. Also, one skilled in the art will appreciate further features and advantages of the disclosure based on the above-described embodiments. Accordingly, the disclosure is not to be limited by what has been particularly shown and described, except as indicated by the appended claims.

What is claimed is:

1. A surgical stapling device comprising:
an adapter assembly having a proximal portion and a distal portion, the distal portion supporting a proximal drive assembly including a rotatable drive member and a drive gear;
a tool assembly including a first jaw and a second jaw, the first jaw and the second jaw coupled to each other to facilitate movement of the tool assembly between an unclamped position and a clamped position, the tool assembly including a proximal portion and a distal portion; and
a distal drive assembly including a pivot member, a sprocket, drive plates formed of a rigid material, and a clamp member secured to one of the drive plates, the pivot member secured to the proximal portion of the tool assembly, the drive plates movable in response to rotation of the sprocket between a stacked configuration and a longitudinally aligned configuration to move the clamp member in relation to the tool assembly between retracted and advanced positions.

2. The surgical stapling device of claim 1, wherein the sprocket includes fingers and each of the drive plates defines openings that receive the fingers such that rotation of the sprocket in a first direction causes sequential advancement of the drive plates from the stacked configuration to the longitudinally aligned configuration to advance the clamp member in relation to the tool assembly.

3. The surgical stapling device of claim 1, wherein the pivot member of the distal drive assembly supports a drive plate holder that defines a cavity the receives and supports the drive plates when the drive plates are in the stacked configuration.

4. The surgical stapling device of claim 1, wherein the pivot member defines a longitudinal slot that is longitudinally aligned with an inner-most one of the drive plates when the drive plates are in the stacked configuration.

5. The surgical stapling device of claim 1, wherein the pivot member is pivotably coupled in relation to the proximal drive assembly about an articulation axis, the articulation axis positioned proximally of the drive plates.

6. The surgical stapling device of claim 2, further including first and second articulation cables extending through the adapter assembly and coupled to the pivot member, the first and second articulation cables movable to pivot the tool assembly about the articulation axis.

7. The surgical stapling device of claim 6, wherein the proximal drive assembly includes a distal housing portion that defines a circular cutout, and the pivot member supports a pivot pin that is received within the circular cutout to pivotably couple the pivot member to the proximal drive assembly.

8. The surgical stapling device of claim 7, wherein the first and second articulation cables extend through channels in the proximal drive assembly and the pivot member, and wherein tension in the first and second articulation cables retains the pivot pin within the circular cutout.

9. The surgical stapling device of claim 1, wherein the proximal drive assembly includes proximal gear that is engaged with the drive gear and the distal drive assembly includes a pivot gear that is engaged with the proximal gear such that rotation of the rotatable drive member of the proximal drive assembly causes rotation of the pivot gear of the distal drive assembly.

10. The surgical stapling device of claim 9, wherein the distal drive assembly includes a sprocket gear that is engaged with the pivot gear and the sprocket such that rotation of the rotatable drive member of the proximal drive assembly causes rotation of the sprocket of the distal drive assembly to advance the clamp member in relation to the tool assembly.

11. The surgical stapling device of claim 2, wherein each of the drive plates includes a distally positioned rib and defines a proximally positioned opening, the ribs received within the openings of adjacent plates to couple the adjacent drive plates to each other as the drive plates move from the stacked configuration to the longitudinally aligned configuration.

12. The surgical stapling device of claim 1, wherein each of the drive plates includes an angled distal cam surface that is positioned to engage a proximal surface of an adjacent one of the drive plates to allow the drive plates to move from the longitudinally aligned position to the stacked configuration.

13. The surgical stapling device of claim 12, further including a biasing member supported within the drive plate holder to urge the drive plates towards the sprocket when the drive plates are in the stacked configuration.

14. The surgical stapling device of claim 1, further including a handle assembly, the proximal portion of the adapter assembly coupled to the handle assembly.

15. A surgical stapling device comprising:
an adapter assembly having a proximal portion and a distal portion;
a tool assembly including an anvil and a cartridge assembly, the anvil and the cartridge assembly coupled to each other to facilitate movement of the tool assembly between an unclamped position and a clamped position, the tool assembly including a proximal portion and a distal portion; and
a distal drive assembly secured to the proximal portion of the tool assembly, the distal drive assembly including a clamp member, drive plates, and a pivot member pivotably coupled to the distal portion of the adapter assembly about an articulation axis, the drive plates supported within the pivot member and movable between a stacked configuration and a longitudinally aligned configuration to move the clamp member in relation to the tool assembly between retracted and advanced positions.

16. The surgical stapling device of claim 15, wherein the distal drive assembly includes a sprocket having fingers and each of the drive plates defines openings that receive the fingers such that rotation of the sprocket in a first direction causes sequential advancement of the drive plates from the stacked configuration to the longitudinally aligned configuration to advance the clamp member in relation to the tool assembly.

17. The surgical stapling device of claim 16, wherein the pivot member of the distal drive assembly supports a drive plate holder that defines a cavity the receives and supports the drive plates when the drive plates are in the stacked configuration.

18. The surgical stapling device of claim 15, further including a proximal drive assembly supported on the distal portion of the adapter assembly, the proximal drive assembly including a distal housing portion that defines a circular cutout, the pivot member supporting a pivot pin that is received within the circular cutout to pivotably couple the pivot member to the proximal drive assembly.

19. The surgical stapling device of claim 18, wherein the first and second articulation cables extend through channels in the proximal drive assembly and the pivot member, and wherein tension in the first and second articulation cables retains the pivot pin within the circular cutout.

20. A surgical stapling device comprising:
an adapter assembly having a proximal portion and a distal portion, the distal portion supporting a proximal drive assembly including a rotatable drive member and a drive gear;
a tool assembly including first jaw and a second jaw, the first jaw and the second jaw coupled to each other to facilitate movement of the tool assembly between an unclamped position and a clamped position, the tool assembly including a proximal portion and a distal portion; and
a distal drive assembly including a pivot member, a sprocket, drive plates formed of a rigid material, and a clamp member secured to one of the drive plates, the pivot member secured to the proximal portion of the tool assembly, the drive plates movable in response to rotation of the sprocket between a stacked configuration and a longitudinally aligned configuration to move the clamp member in relation to the tool assembly between retracted and advanced positions;
